(19) United States
(12) Patent Application Publication
(10) Pub. No.: US 2009/0118363 A1
(43) Pub. Date: May 7, 2009

(54) SESAMIN-CLASS COMPOUND-CONTAINING FOODS OR BEVERAGES FOR ANIMALS

(75) Inventors: Yukihiro Aoshima, Osaka (JP); Junko Tsuji, Tokyo (JP)

Correspondence Address: DRINKER BIDDLE & REATH (DC), 1500 K STREET, N.W., SUITE 1100, WASHINGTON, DC, 20005-1209, US

(73) Assignee: SUNTORY LIMITED, Osaka-shi, Osaka (JP)

(21) Appl. No.: 12/295,833

(22) PCT Filed: Apr. 3, 2007
(86) PCT No.: PCT/JP2007/057461

§ 371 Date: Oct. 2, 2008

(30) Foreign Application Priority Data
Apr. 3, 2006 (JP) 2006-102312

(51) Int. Cl. A61K 8/49; A61Q 5/00; A61Q 15/00

(52) U.S. Cl. 514/464

(57) ABSTRACT

Methods for treating coats and/or body odors of animals are provided. Foods and beverages for nonhuman animals that can be used in the methods are provided.

Foods and beverages containing a sesamin-class compound(s) of 1 wt % or above and an additive acceptable as a food or beverage are provided. Methods for treating coats and/or body odors of nonhuman animals comprising administering to the nonhuman animals foods or beverages for nonhuman animals containing an effective amount of the sesamin-class compound(s) needed to treat the coats and/or the body odors of the nonhuman animals are provided. Preferably, the sesamin-class compound(s) are dissolved in fat or oil.

### TECHNICAL FIELD

The present invention relates to sesamin-class compound-containing foods and beverages for nonhuman animals. The present invention also relates to methods for treating coats and/or body odors of nonhuman animals, which methods include administering to the nonhuman animals the foods or beverages for nonhuman animals.

### BACKGROUND ART

In recent years, pets such as dogs and cats are treated as a family member, and pet owners increasingly care about health condition of their pets. Changes in coats of pets can be noticed from their appearance, and changes in body odors of pets can be recognized by smelling the body odors. Thus, the changes in coats and body odors are used as important barometers for pet owners to find out the health condition of their pets. For instance, it is known that coat condition strongly links to skin diseases, which are the most common cause for visiting animal hospitals. If pets are not in good health condition because of skin diseases or the like, their coats are likely to deteriorate in condition, such as coat gloss and coat texture. Further, the body odors of pets are produced mainly by waste products that are secreted by sweat glands of the pets and attached to skin of the pets. If pets are not in good health condition, the body odors of the pets are likely to become stronger. Further, many pets are kept indoors nowadays, so that rooms are filled with the body odors or the like of pets. Thus there are many pet owners who care about the odors of pets, compared with cases in which pets are kept outdoors. Accordingly, reducing pet odors has been demanded because of the foregoing circumstances.

Diet therapy is known as an effective tool for improving the condition of pet coats and reducing the body odors of pets, and various types of foods and beverages that possess actions to improve the coats and to reduce the body odors are suggested. For instance, foods and beverages by which the coat condition of animals, such as coat gloss and coat texture, may be improved are suggested in Patent Literatures-1-3. Patent Literature 1 describes a rabbit feed by which gloss of rabbit hair improves. The rabbit feed is produced by adding carrot juice and fish protein degradation peptides to a feed that contains mainly vegetable materials. Patent Literature 2 describes a pet food containing Grifola frondosa powders and/or Grifola frondosa extracts to improve shine of pet coats. Patent Literature 3 describes a diet and a foodstuff that contain zinc and linoleic acid at specific concentrations to enhance and/or improve condition of skins and coats of animals. Further, supplements for pets to maintain pet health are also suggested; Patent Literature 4 mentions that supplements for pets that are in paste form and contain beer yeast, highly-unsaturated fatty acid, collagen peptide, keratin hydrolysate, zinc, and vitamins, are effective in improving the condition of pet coats such as deteriorated coat gloss and coat texture.

Sesamin-class compounds, on the other hand, are known to possess various physiological activities. Patent Literature 5 describes a food and a beverage that contain sesamin and/or episesamin. Patent Literature 6 describes a body fat reducing agent that contains a specific dioxabicyclo[3.3.0]octane derivative as an active ingredient, and mentions that a 0.2% sesamin-class compound-containing feed produces an effect of reducing body fat of rats. None of the patent literatures, however, suggests or discloses an effect of a sesamin-class compound(s) on the coats or the body odors of animals.

Further, Patent Literature 7 discloses a feed in a form of a soft capsule filled with fat or oil. However, the capsule is to supply the fat or the oil as nutrients, and Patent Literature 7 neither suggests nor discloses an effect on the coats or the body odors.

### [Patent Literature 1] Japanese Unexamined Patent Publication No. 38070/1996 (Tokukaihei 8-38070)

### [Patent Literature 2] Japanese Unexamined Patent Publication No. 38069/1996 (Tokukaihei 8-38069)

### [Patent Literature 3] Published Japanese Translation of PCT International Publication for Patent Application, Publication No. 504816/2002 (Tokuhyo 2002-504816)

### [Patent Literature 4] Japanese Unexamined Patent Publication No. 357508/2004

### [Patent Literature 5] Japanese Patent No. 3001589

### [Patent Literature 6] Japanese Patent No. 3205315

### [Patent Literature 7] Japanese Unexamined Patent Publication No. 186253/1985

### DISCLOSURE OF THE INVENTION

### Problems to be Solved by the Invention

As discussed above, although various methods for improving animal coats and reducing animal body odors have been suggested, there has been no method that is sufficiently effective. Furthermore, many of active ingredients contained in the foods and beverages that possess the actions to improve the coats and to reduce the body odors have distinctive flavors, and this in many cases makes the foods and beverages unpalatable to animals to discourage the animals from ingesting (eating/drinking) them, and further affects a smell of stool and a smell of urine.

### Means for Solving the Problems

The inventors of the present invention diligently studied to solve the above problems. As a result, they found that sesamin-class compounds isolated from sesame seeds, sesame residues, and sesame oils were effective in improving the coats of the pets and in reducing the body odors of the pets. They also found that, surprisingly, those effects would be enhanced if the sesamin-class compounds dissolved in fat or oil were ingested (eaten/drunk). The inventors confirmed that, since isolated and purified sesamin and/or episesamin were tasteless, odorless, and white, they were suitable to be added to the foods and beverages and would not affect smells of stool or urine in the cases in which they were ingested. With this confirmation, the inventors completed the present invention.

Specifically, the present invention provides a food or beverage for a nonhuman animal, comprising: a sesamin-class compound(s) of 1 wt % or above, and an additive acceptable as a food or beverage for an animal. The present invention further provides a food or beverage for a nonhuman animal to treat a coat and/or a body odor of the nonhuman animal, the food or beverage comprising: an effective amount of a sesamin-class compound(s) needed to treat the coat and/or the body odor of the nonhuman animal, and an additive acceptable as a food or beverage for an animal.

The present invention further provides the above-specified food or beverage, wherein the sesamin-class compound(s) are sesamin and/or episesamin, preferably isolated and purified sesamin and/or episesamin. The present invention further provides the above-specified food or beverage further comprising a fat or an oil; it is preferable that the food or beverage comprise the sesamin-class compound(s) dissolved in the fat or the oil.

The present invention further provides the food or beverage each of which is in a form of a supplement, preferably a soft capsule, more preferably a soft capsule having a longer diameter of 8.5-14.5 mm and a shorter diameter of 6.0-9.0 mm.

As used herein, the “sesamin-class compound” includes sesamin and its analogs, specifically sesamin, sesaminol, episesaminol, sesamolin, episesamin, dioxabicyclo [3.3.0] octane derivatives (e.g., those described in Japanese Unexamined Patent Publication No. 9331/1992 (Tokukaihei 04-9331)), etc. Especially, sesamin and episesamin are preferred in the present invention. Moreover, the “sesamin-class compound” also includes glycosides of sesamin and its analogs as well as their metabolites, and any of them may be contained in the food and beverage for nonhuman animals of the present invention, and in the food and beverage for nonhuman animals in the methods of the present invention (hereinafter, they are sometimes referred to simply as “the present food and beverage”) so far as they are suitable for providing the food and beverage and the method of the present invention, either alone or in combination. Alternatively, it is also possible to add the sesamin-class compounds contained in extracts from natural products (sesamin-class compound-containing extract).

Neither the sesamin-class compound(s) nor the sesamin-class compound-containing extract is limited by the forms, the preparation process or the like. For example, those extracted from sesame oil by a publicly known method (e.g., extracting sesame oil with hot methanol, and after removal of methanol, extracting the residue with acetone, as described in Japanese Unexamined Patent Publication No. 9331/1992 (Tokukaihei 04-9331)) can be used, or commercially available sesame oil (in liquid form) (e.g., sesame oil having sesamin concentrated by supercritical extraction) may be used as such, but it is important to be careful about a sesamin-class compound content described below. The sesame oil used as raw material is not specifically limited. Note that if the sesame oil or the sesamin-class compound-containing extract of the sesame oil has a roast flavor of sesame, the flavor of sesame can be added by using the sesame oil or the sesamin-class compound-containing extract in the present food and beverage.

However, since the flavor specific to sesame oil is sometimes evaluated as organoleptically unfavorable, it is preferable to use the sesamin-class compound(s) or the sesamin-class compound-containing extract that is tasteless and odorless and is extracted from sesame oil, more preferably the sesamin-class compound(s) or the sesamin-class compound-containing extract that is isolated and purified. Further, in a case in which a desired amount of sesamin-class compound(s) needed to effectively treat the coats or the body odors is to be included by using sesame oil as it is, the sesamin-class compound content in the sesame oil is so low that the volume of the resulting food and beverage of the present invention may excessively increase to cause difficulty in consuming. Especially, formulations for oral administration (tablets, capsules, etc.) become bulky so as to cause difficulty in consuming. Thus, the sesamin-class compound(s) that are isolated and purified are preferably used because it may be taken in low doses.

In the present invention, the present food and beverage produce remarkable effects of improving the coats and/or reducing the body odors if containing the sesamin-class compound(s) that are dissolved in fat or oil. Thus, the present food and beverage may contain sesame oil, sesamin-class compound-containing extracts (sesame oil concentrates) from sesame oil in which the sesamin-class compound(s) are dissolved, or the like. In a case in which powdery or solid sesame extracts, powdery or solid sesamin-class compounds that are isolated and purified, or the like are to be used, it is preferable that they be included together with fat or oil, and the present food and beverage contain the sesamin-class compound(s) that are dissolved in the fat or oil.

The fat or oil is not particularly limited, as long as they are edible oil that can be orally ingested and allows the sesamin-class compound(s) to dissolve. Examples of the fat or oil that can be used include natural animal fats and natural vegetable oils such as soybean oil, canola oil, safflower oil, olive oil, cotton seed oil, corn oil, palm oil, rice bran oil, wheatgerm oil, sunflower seed oil, saffron thistle oil, peanut oil, whale oil, sardine oil, and cuttlefish oil, medium-chain triglyceride (MCT), mono-, di- or tri-glyceride with a fatty acid(s) having 4-22 carbon atoms, and mixtures thereof. Further, the fat or oil is not limited to those in liquid form at ambient temperature; those in semi-solid or solid forms can also be used, such as lard, beef fat, hydrogenated fish oil, margarine, shortening, and mixtures thereof, and, furthermore, it is also possible to use them in combination with the liquid fat or oil. Since the sesamin-class compound(s) and the sesamin-class compound-containing extract are originally contained in fat or oil, they easily dissolve in fat or oil, and it is possible to dissolve them into fat or oil by mixing them with the fat or oil at ambient temperature. If necessary, however, heating or the like may be conducted.

The amount of the fat or oil in the present food and beverage containing the sesamin-class compound(s) and the fat or oil is determined so as to allow the sesamin-class compounds to dissolve in the fat or oil at least partially, preferably completely. As described in Reference Example below, the Applicant of the present application found that solubility of the sesamin-class compound(s) in fat and oil varied according to the type of the fat or oil (refer to Reference Example, Table 1). Accordingly, the solubility in a desired fat or oil of the sesamin-class compound(s) that are to be used is taken into consideration to determine the amount of the sesamin-class compound(s) to be contained and the amount of the fat or oil to be contained. For instance, the fat or oil approximately 15-200 (preferably 50-100) times (ratio by weight) the total amount of the sesamin-class compound(s) are to be contained. Note that there would be substantially no upper limit on the ratio of the fat or oil to the sesamin-class compound(s) if administration of the fat or oil as nutrition is taken into consideration.

The term “treating coats” of animals as used herein refers to treatments carried out to improve condition of coats of animals to a level at which improvement can be recognized from appearance, treatments carried out to prevent condition of the coats from deteriorating, or treatments carried out to keep fine condition of the coats. Persons skilled in the art easily determine by, for instance, looking at coat gloss and coat texture of animals, as described in Examples below.

Further, the term “treating body odors” of animals as used herein refers to treatments carried out to reduce body odors, treatments carried out to prevent body odors from becoming severer, treatments carried out to maintain light body odors at the same level, treatments carried out to change body odors that are unpleasant to odors that are not unpleasant, treatments carried out to prevent body odors from changing to unpleasant odors, or treatments carried out to maintain body odors that are not unpleasant. Persons skilled in the art easily determine changes in body odors.

Especially if the animals are pets, owners of the pets are the ones who are most sensitive to changes in condition of the coats and the body odors of the pets. Thus, judgment of pet owners is most reliable in evaluation of the treatments of the coats and the body odors of the pets.

Nonhuman animals as used herein refer to animals other than humans, preferably animals that may have problems of condition of the coats or the body odors, specifically industrial animals, pets, and laboratory animals. The industrial animals refers to those animals which are required to be kept for industrial purposes, including livestock such as cattle, horses, pigs, goats, sheep, etc., poultry such as chickens, ducks, quails, turkeys, ostriches, etc.; pets refers to so-called companion animals, including dogs, cats, rabbits, marmots, little birds, hamsters, etc.; and laboratory animals refers to those animals which are sacrificed for research in such disciplines as medicine, biology, agronomy, pharmacy, etc. and they include rats, guinea pigs, beagles, miniature pigs, rhesus monkeys, crab-eating monkeys, etc. Among those listed above, nonhuman mammals are most preferred as targets to which the present food and beverage are to be administered.

Among the nonhuman animals above, the pets are the animals that are expected the most to undergo the treatments of the coats and the body odors. Especially body odors of dogs and cats are relatively easy to notice because they mainly eat animal proteins such as meat and fish to cause odorous gases to be produced easily in stomach, intestines, and oral cavity. Thus, cats and dogs are preferred targets for administration of the present food and beverage. Concrete examples of the dogs include Beagle, Sheltie, Pomeranian, Shih-Tzu, Shiba-Inu, Spits, Cavalier, Miniature Dachshund, Labrador Retriever, West Highland White Terrier, and a mix; the dogs, however, are not limited to those listed above. Concrete examples of the cats include Chinchilla, Scottish Fold, Persian cats, American Shorthair, Abyssinian cats, and a mix; the cats, however, are not limited to those listed above.

The term “food and beverage for animals” as used herein refers to feeds, pet foods, confectioneries for animals (e.g., jelly, biscuits), canned foods for animals such as sausages and salamis, supplements for pets, and beverages; they are sometimes referred to as “pet feed” herein. The food and beverage for animals further encompass pharmaceutical compositions for oral administration. The food and beverage for animals can be prepared in any form, including solid forms (e.g., powder, granule, tablet, capsule), semi-solid forms (e.g., gel, paste), and liquid forms (e.g., solution, emulsion, dispersion).

The present invention provides methods for treating coats and/or body odors of animals. The methods include administering to nonhuman animals the food and beverage for animals. Specifically, the present food and beverage for animals effectively prevent condition of coats from deteriorating and prevent body odors, if administered on a regular basis. Further, it is known that the sesamin-class compound(s) contained in the present food and beverage for animals possesse various physiological activities as well as the treatments described above. Thus, the present food and beverage are also expected to maintain or enhance animal health if administered on a continuous basis. To administer on a continuous basis, the forms described above such as feeds and pet foods are employable, but the form of a supplement such as capsules (e.g., soft capsules) and tablets is preferred because it enables adjustment of the amount to be administered, and stable and reliable administration.

Further, it is preferable in the present invention that the present food and beverage contain the sesamin-class compound(s) dissolved in the fat or oil. However, since the fat or oil oxidizes and deteriorates easily, administering without knowing this fact may harm the health of the animals. Further, although the sesamin-class compound(s) possesse an activity to reduce radicals such as active oxygen, its action is so weak that the fat or oil would not be sufficiently prevented from oxidizing and deteriorating. Furthermore, if the sesamin-class compound(s) prevent the fat or oil from oxidizing and deteriorating, i.e., if the sesamin-class compounds) act as an antioxidant, in vivo effect of the present invention may be reduced. In view of the foregoing points, it is preferable to administer in the form of a supplement (form by which the fat or oil can be prevented from oxidizing and deteriorating) containing either the sesamin-class compound(s) alone or a combination of the sesamin-class compound(s) and the fat or oil, in order to produce the effect of the present invention.

Capsules are especially preferred among the supplements in the cases in which the fat or oil is to be contained, because, with the form of a capsule, the fat or oil is better prevented from oxidizing and deteriorating. A capsule containing liquid (e.g., cases in which the sesamin-class compound(s) are dissolved in liquid fat or liquid oil at ambient temperature) is normally in the form of a soft capsule. The capsules may be used as supplements to administer the sesamin-class compounds, supplements further containing other ingredients to supplement or to furnish nutrition, or dietary supplements for the purposes above.

The present food and beverage contain additives acceptable as food and beverage for animals. The additives are those commonly used in food and beverage for animals, and examples of the additives include vitamins such as vitamin E, vitamin C; sugars, excipients, disintegrants, binders, lubricants, emulsifiers, tonicity agents (isotonizing agents), buffers, solubilizers, preservatives, stabilizers, antioxidants, colorants, flavoring agents, perfumes, coagulants, pH-modifiers, thickeners, extract powders, herbal medicines, inorganic salts, etc. The additives, however, are not limited to those listed above, as long as the desired effect of the sesamin-class compound(s) are not vitiated. Especially if the present food and beverage are the supplements, for example vitamins, such as vitamin E and vitamin C, and/or those commonly used to prepare the supplements, such as emulsifiers, tonicity agents (isotonizing agents), buffers, solubilizers, preservatives, stabilizers, and antioxidants, may be contained as appropriate.

Further, the additives are preferably those acceptable as food and beverage for nonhuman animals, specifically additives that nonhuman animals like to eat or drink. For instance, nonhuman animals like to eat or drink some kinds of additives that are not sufficiently purified for humans to eat or drink and have odors and tastes that humans do not like. More specifically, for instance if gelatin is to be used as the additive, gelatins that are less purified are sometimes preferred because they have animal odors that the nonhuman animals such as dogs and cats like, and are inexpensive. To make the animals eat the present food and beverage voluntarily, flavors and/or tastes that the animals like, such as meat flavor and fish flavor, may be added to the inside or onto a surface of the present food and beverage. A method for adding to a surface of food and beverage (especially preparations such as supplements) a flavor that the animals like is found in, for instance, Japanese Unexamined Patent Publication No. 2004-194514, but the method is not limited to that described in this publication.

The food and beverage for animals can be prepared by persons skilled in the art using publicly known methods. For instance, the present food and beverage in the form of capsules can be prepared by publicly-known encapsulation techniques. The soft capsules, for instance, can be prepared using gelatin or polysaccharide as a film-forming agent. An additive that is commonly used in preparation of soft capsules, such as sorbitol and glycerine, may be added to the gelatin or the polysaccharide. Exemplary methods for preparing the soft capsules include an immersion method including molding capsules and at the same time filling the capsules with a content fluid, a punching method, and an instillation method.

In a case in which a commonly-used punching method is employed, a gelatin (or polysaccharide) solution is flattened thin, cooled, and then turned into gel, and thereafter gelatin (or polysaccharide) sheets are formed. Then, a prepared fluid is injected between two gelatin (or polysaccharide) sheets, and they are pressed from both sides with a mold. A sesamin-class compound(s) added to fat or oil are injected into capsules. This is immediately followed by heating the gelatin (or polysaccharide) sheets to let them adhere, and punching them. Thereafter, they are dried to produce the soft capsules which are the present food and beverage.

Neither shape nor size of the present food and beverage is limited, and the present food and beverage can have any shape and size within a usual range in the technical field. To use the present food and beverage in the methods of the present invention, the present food and beverage may be prepared to have a shape that is easy to administer to the nonhuman animals that are the targets for the administration of the present food and beverage. In general, animals have a habit of biting an object when eating it. If an animal bites the present food and beverage (e.g., soft capsule) containing liquid fat or liquid oil, the fat or oil may spill out of a mouth of the animal to make a smudge around the mouth. It is thus preferable that the shape have a size that does not require the animals to bite, or a size determined such that the liquid fat or liquid oil would not spill out of a mouth of an animal when the animal bites the present food and beverage to eat. Concretely, a soft capsule having a longer diameter of approximately 8.5-14.5 mm and a shorter diameter of approximately 6.0-9.0 mm is preferred in the cases in which the present food and beverage are in the form of the soft capsules and are to be administered to the nonhuman animals which are dogs.

The present food and beverage for animals are the sesamin-class compound(s) or the sesamin-class compound-containing extract, including those in solid form that are mixed with the pet feed, and those in liquid form that are sprayed to the pet feed; commercially-available products may be used as the pet feed. If the present food and beverage are in the form of supplements such as soft capsules, it is also possible to use them by adding them to feed, pet food, or the like.

Note that concrete purposes of use (e.g., to improve condition of coats (e.g., coat gloss, coat texture), to maintain condition of coats, to prevent condition of coats from deteriorating, to improve body odors, to maintain fine condition of body odors, to prevent body odors from deteriorating, to furnish nutrition, to supplement nutrition, to maintain health) and/or concrete directions for use (e.g., amount of administration, number of administration, way of administration) may be indicated on the present food and beverage.

The present invention provides methods for treating coats and/or body odors of nonhuman animals, which methods include administering to the nonhuman animals the above-described food and beverage for the nonhuman animals.

The present invention further provides methods for treating coats and/or body odors of nonhuman animals, which methods include administering to the nonhuman animals an effective amount of the sesamin-class compound(s) needed to treat the coats and/or the body odors of the nonhuman animals; the sesamin-class compound(s) are preferably contained in the food and beverage for the nonhuman animals when administered to the nonhuman animals.

The present invention further provides the methods above, in which the effective amount is not less than 0.15 mg, preferably not less than 0.19 mg, per 1 kg of body weight of the respective nonhuman animals per day. The present invention further provides the methods above, in which the nonhuman animals are pets.

The effective amount of the sesamin-class compound(s) needed to treat the coats and/or the body odors of the nonhuman animals in the methods of the present invention is normally 0.1-10 mg, preferably 0.15-5 mg, per 1 kg of body weight of the respective nonhuman animals per day, but it varies according to the type and size of target animals, the condition of coats and/or body odors, health condition, and the like. If the purpose of the methods is to improve condition of coats or body odors, the effective amount is sometimes greater than that in the cases in which the purpose is either to maintain fine condition of the coats and/or the body odors or to prevent condition of the coats and/or the body odors from deteriorating. Note that the inventors of the present invention confirmed the coat improving action and the body odor reducing action when the pets (dogs) ingested 0.19 mg of the sesamin-class compound(s) per 1 kg of body weight of the respective pets (dogs) per day (refer to Example 4). Persons skilled in the art can refer to, for instance, the above-specified effective amounts to determine an appropriate amount of the sesamin-class compound(s) to be contained and an appropriate amount of the fat or oil to be contained in the present food and beverage according to the form of the food and beverage for administration.

Persons skilled in the art can select appropriate periods and methods of administration of the present food and beverage and administration of the sesamin-class compound(s) in the method of the present invention according to the type, size, condition of coats and/or body odors, health condition, and the like of the target animal.

For instance if the purpose is to improve the condition of coats or body odors, the amount specified in Examples below, for instance, may be administered using a reliable administration method (e.g., a single supplement per day) for a period of 3 days to 8 weeks, preferably 1-6 weeks, more preferably 2-4 weeks.

Further, for instance if the purpose is either to maintain fine condition of coats and/or body odors or to prevent conditions of coats and/or body odors from deteriorating, a longer period of administration on a continuous basis than that in the cases in which the purpose is to improve the condition is preferred; for instance if the present food and beverage is to be added to feed, pet food, or the like, an administration method that is suitable for the administration on a continuous basis is employed.

### BRIEF DESCRIPTION OF THE DRAWINGS

FIG. 1 is a bar chart showing changes in body odor after dogs ingested the present food and beverage in Example 2.

FIG. 2 is a bar chart showing changes in coat gloss after the dogs ingested the present food and beverage in Example 2.

FIG. 3 is a bar chart showing changes in coat texture after the dogs ingested the present food and beverage in Example 2.

FIG. 4 is a bar chart showing changes in body odor two weeks after and four weeks after dogs ingested the present food and beverage in Example 3.

FIG. 5 is a bar chart showing changes in coat gloss two weeks after and four weeks after the dogs ingested the present food and beverage in Example 3.

FIG. 6 is a bar chart showing changes in coat texture two weeks after and four weeks after the dogs ingested the present food and beverage in Example 3.

### EXAMPLES

The following examples further illustrate the present invention without, however, limiting the invention thereto.

### Reference Example 1

Test of Dissolving Sesamin-Class Compounds into Fats and Oils

Solubility of sesamin-class compounds to various fats and oils was determined. Sesamin, episesamin, and a mixture of sesamin and episesamin, sesamin:episesamin=51.1:48.2 (Takemoto Oil and Fat Co., Ltd.), were used as the sesamin-class compounds. The sesamin and the episesamin were prepared by isolating them from the mixture and purifying them. The respective sesamin-class compounds were dissolved into five different kinds of fats and oils, 100 g each, shown in Table 1 to determine a limit amount (solubility) of the respective sesamin-class compounds that can be dissolved completely. The results are shown in Table 1. It is understood from Table 1 that the sesamin, the episesamin, and the mixture of the sesamin and the episesamin have different solubilities, and that the solubility varies in a manner that depends on the types of the fats and oils.

TABLE 1
Solubility of Sesamin, Episesamin, and Mixuture of
Sesamin and Episesamin in Various Fats and Oils (%)

| FATS AND | WHEATGERM | OLIVE | MCT- | MCT- | |
|---|---|---|---|---|---|
| OILS | OIL | OIL | 1*¹ | 2*² | DG*³ |
| MIXTURE*⁴ | 2.0 | 1.5 | 7.0 | 6.5 | 1.5 |
| SESAMIN | 0.75 | 0.75 | 4.0 | 2.0 | 1.25 |
| EPISESAMIN | 0.75 | 0.5 | 2.5 | 2.5 | 1.0 |

*¹MCT-1: “Actor M-1” of Riken Vitamin Co. Ltd. (medium-chain triglyceride of C8:C12 = 1:1)
*²MCT-2: “Actor M-2” of Riken Vitamin Co. Ltd. (C8 medium-chain triglyceride)
*³Diacylglycerol: “Econa Cooking Oil” of Kao Corporation
*⁴Mixture: sesamin:episesamin = 51.1:48.2

### Example 1

### Preparation of Sesamin-Class Compound-Containing Food and Beverage for Nonhuman Animals

A mixture of powdery sesamin and powdery episesamin (sesamin:episesamin=51.1:48.2 (weight ratio), Takemoto Oil & Fat Co., Ltd.) isolated from sesame oil and purified (hereinafter, the mixture is sometimes referred to as “sesamin-class compound mixture”) was used as the sesamin-class compounds. A tablet, soft capsule A, and soft capsule B containing ingredients as shown in Table 2 were prepared. Concretely, raw materials of the tablet were mixed at a predetermined proportion and then punched by a single punch tableting to prepare tablets each having a diameter of 11 mm and a weight of 330 mg. Further, raw materials of the capsules were mixed at a predetermined proportion and then filled into capsule films (74.0% gelatin, 26.0% glycerine, an appropriate amount of water) by a conventional method (rotary method) to prepare soft capsules A each having a longer diameter of 14.2 mm, a shorter diameter of 6.6 mm, and a weight of 360 mg, and soft capsules B each having a longer diameter of 9.5 mm, a shorter diameter of 6.7 mm, and a weight of 250 mg.

TABLE 2
(Tablet)

| | | |
|---|---|---|
| SESAMIN-CLASS COMPOUND MIXTURE | 5 | mg |
| VITAMIN E | 30 | mg |
| EXCIPIENT | 295 | mg |

(Soft Capsule A)

| | | |
|---|---|---|
| SESAMIN-CLASS COMPOUND MIXTURE | 3.4 | mg |
| VITAMIN E | 20 | mg |
| WHEATGERM OIL | 176.6 | mg |

(Soft Capsule B)

| | | |
|---|---|---|
| SESAMIN-CLASS COMPOUND MIXTURE | 1.7 | mg |
| VITAMIN E | 9.5 | mg |
| WHEATGERM OIL | 138.8 | mg |

### Example 2

### Test (1) of Oral Ingestion of Sesamin-Class-Compound-Containing Food and Beverage for Nonhuman Animals by Dogs

Ten dogs were divided into two groups each having five dogs. Each of the groups ingested for four weeks either the tablet or soft capsule A prepared in Example 1, in the amount specified in Table 3 (the group to which the tablets were given are referred to as a “tablet group”, and the group to which the soft capsules were given are referred to as a “capsule group”). Changes that were not obtained by normal meals of the dogs were evaluated. Table 3 shows information on the respective dogs (type, age, weight), the number of tablets or capsules administered, and normal meals of the respective dogs.

TABLE 3
(Tablet Group)

| No. | P-1 | P-2 | P-3 | P-4 | P-5 |
|---|---|---|---|---|---|
| TYPE OF DOG | BEAGLE | SHELTIE | POMERANIAN | SHIH-TZU | SHIBA-INU |
| AGE (YEARS) | 13 | 7 | 9 | 10 | 12 |
| BODY WEIGHT | 14 | 13 | 9 | 7 | 10 |
| (KG) | | | | | |
| NUMBER OF | 2 | 2 | 2 | 2 | 2 |
| TABLETS | | | | | |
| ADMINISTERED | | | | | |
| (PIECES) | | | | | |
| TYPE OF | COMMERCIALLY- | COMMERCIALLY- | COMMERCIALLY- | COMMERCIALLY- | COMMERCIALLY- |
| NORMAL MEAL | AVAILABLE | AVAILABLE | AVAILABLE | AVAILABLE | AVAILABLE |
| | DOG FOOD | DOG FOOD | DOG FOOD | DOG FOOD | DOG FOOD |
| | | | JERKY | | |

(Capsule Group)

| No. | S-1 | S-2 | S-3 | S-4 | S-5 |
|---|---|---|---|---|---|
| TYPE OF DOG | MIX | SHELTIE | SPITZ | BEAGLE | SHIBA-INU MIX |
| AGE (YEARS) | 17 | 9 | 14 | 11 | 14 |
| BODY WEIGHT | 12 | 18.6 | 6 | 14 | 10 |
| (KG) | | | | | |
| NUMBER OF | 3 | 3 | 3 | 3 | 3 |
| CAPSULES | | | | | |
| ADMINISTERED | | | | | |
| (PIECES) | | | | | |
| TYPE OF | COMMERCIALLY- | COMMERCIALLY- | COMMERCIALLY- | COMMERCIALLY- | COMMERCIALLY- |
| NORMAL MEAL | AVAILABLE | AVAILABLE | AVAILABLE | AVAILABLE | AVAILABLE |
| | DOG FOOD | DOG FOOD | DOG FOOD | DOG FOOD | DOG FOOD |
| | JERKY | | | BISCUITS | |

A questionnaire survey of the body odors and coats (coat gloss, coat texture) was conducted with pet owners. Condition before the ingestion and condition four weeks after the ingestion were evaluated on a five-point scale from +2 to −2. Concrete ways of the evaluation were as follows:

Body odor

| | |
|---|---|
| None | +2 |
| Weak | +1 |
| Average | 0 |
| Slightly strong | −1 |
| Strong | −2; |

Coat gloss

| | |
|---|---|
| Glossy | +2 |
| Slightly glossy | +1 |
| Average | 0 |
| Slightly matt | −1 |
| Matt | −2; and |

Coat texture

| | |
|---|---|
| Very good | +2 |
| Good | +1 |
| Average | 0 |
| Poor | −1 |
| Very poor | −2. |

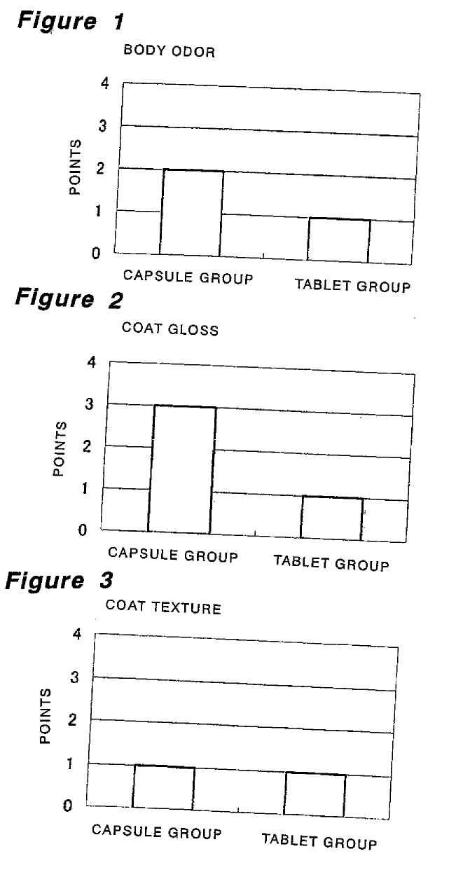

An amount of change (=points obtained four weeks after the ingestion—points obtained before the ingestion) in the respective categories, including the body odor, the coat gloss, and the coat texture, was calculated for the respective dogs, and a sum of the points of all dogs was calculated for each of the groups; a total of the amount of change was used as a point for the respective groups. FIGS. 1-3 show the points earned in the respective categories, including the body odor, the coat gloss, and the coat texture. The points were positive in all of the categories, including the body odor, the coat gloss, and the coat texture. This indicates that the ingestion of the sesamin-class compound-containing food and beverage for nonhuman animals reduces the body odors and improves the condition of coats. Furthermore, it is understood from the results of the body odors and the coat gloss as shown in FIGS. 1 and 2 that better effect was produced in the capsule group than in the table group. This indicates that the ingestion of the sesamin-class compound(s) dissolved in the fat or oil produces better effect.

Note that properties of stool (hardness, odor, quantity) were observed during the test of oral ingestion, and there was no change in the properties compared with those of normal stool. This indicates that neither the ingestion of the tablets nor the ingestion of the capsules caused loose stool, specific odor, or changes in the intake of food.

### Example 3

### Test (2) of Oral Ingestion of Sesamin-Class Compound-Containing Food and Beverage for Nonhuman Animals by Dogs

Seven dogs shown in Table 4 ingested for four weeks soft capsules A prepared in Example 1, in an amount appropriate for body weight of each dog as shown in Table 4. Changes that were not obtained by normal meals of the dogs were evaluated. The body odor, the coat gloss, and the coat texture were evaluated in the same manner as described in Example 2 before the ingestion, two weeks after the ingestion, and four weeks after the ingestion. Points were calculated using the same method as that described in Example 2.

TABLE 4

| No. | S-1 | S-2 | S-3 | S-4 |
|---|---|---|---|---|
| TYPE OF DOG | MIX | CAVALIER | MINIATURE | LABRADOR |
| | | | DACHSHUND | RETRIEVER |
| AGE (YEARS) | 12 | 13 | 8 | 7 |
| BODY WEIGHT (KG) | 13 | 8 | 5 | 24 |
| NUMBER OF CAPSULES | 3 | 3 | 3 | 6 |
| GIVEN (PIECE) | | | | |
| TYPE OF NORMAL | COMMERCIALLY- | COMMERCIALLY- | COMMERCIALLY- | COMMERCIALLY- |
| MEAL | AVAILABLE | AVAILABLE | AVAILABLE | AVAILABLE |
| | DOG FOOD | DOG FOOD | DOG FOOD | DOG FOOD |
| | JERKY | COOKIES | JERKY | |

| No. | S-5 | S-6 | S-7 |
|---|---|---|---|
| TYPE OF DOG | MINIATURE | WEST HIGHLAND | YORKSHIRE |
| | DACHSHUND | WHITE TERRIER | TERRIER |
| AGE (YEARS) | 6 | 10 | 9 |
| BODY WEIGHT (KG) | 5.8 | 9 | 2.9 |
| NUMBER OF CAPSULES | 3 | 3 | 3 |
| GIVEN (PIECE) | | | |
| TYPE OF NORMAL | COMMERCIALLY- | COMMERCIALLY- | COMMERCIALLY- |
| MEAL | AVAILABLE | AVAILABLE | AVAILABLE |
| | DOG FOOD | DOG FOOD | DOG FOOD |
| | BISCUITS | CHICKEN | BISCUITS |
| | | FILLET | |

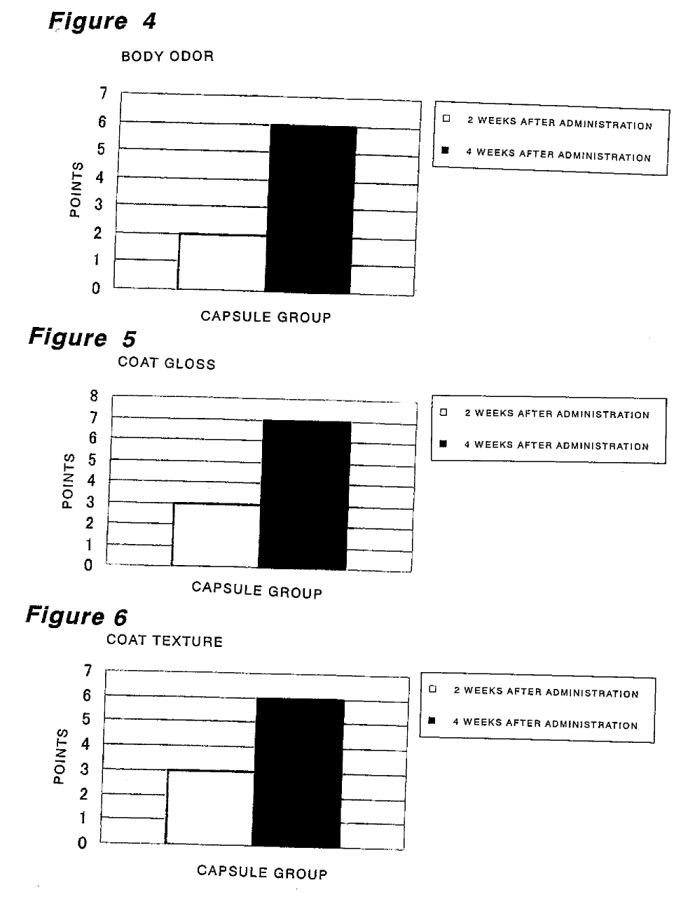

FIGS. 4-6 show the points earned in the body odor, the coat gloss, and the coat texture before and after the ingestion of the sesamin-class compound-containing food and beverage. It was confirmed that oral ingestion of the sesamin-class compound-containing food and beverage (capsules) for nonhuman animals, which sesamin-class compound(s) are dissolved in fat or oil, resulted in improvement over time in all of the categories, including the body odor, the coat gloss, and the coat texture, in the same manner as in Example 2. Note that properties of stool (hardness, odor, quantity) were observed during the test of oral ingestion, and there was no change in the properties compared with those of normal stool.

### Example 4

### Test (3) of Oral Ingestion of Sesamin-Class Compound-Containing Food and Beverage for Nonhuman Animals by Dogs

A dog, which was a three-year-old Shiba-Inu with the body weight of 9 kg, ingested soft capsule B prepared in Example 1, a single capsule per day for four weeks. Changes that were not obtained by a normal meal of the dog were evaluated.

The results are shown in Table 5. Changes in body odor, coat gloss, and coat texture were observed before and after the ingestion, and improvement was observed in all categories after the ingestion of the capsule. This indicates that the ingestion of the sesamin-class compound(s) in such a small amount of 0.19 mg per 1 kg of body weight of the dog per day also produces the effect of reducing the body odor and the effect of improving the coat.

TABLE 5

| | BODY | COAT | COAT |
|---|---|---|---|
| | ODOR | GLOSS | TEXTURE |
| BEFORE | AVERAGE | AVERAGE | AVERAGE |
| INGESTION | | | |
| 1 WEEK AFTER | AVERAGE | (NOT | (NOT |
| INGESTION | | OBSERVED) | OBSERVED) |
| 2 WEEKS AFTER | AVERAGE | SLIGHTLY | AVERAGE |
| INGESTION | | BETTER | |
| 3 WEEKS AFTER | AVERAGE | (NOT | (NOT |
| INGESTION | | OBSERVED) | OBSERVED) |
| 4 WEEKS AFTER | SLIGHTLY | SLIGHTLY | SLIGHTLY |
| INGESTION | WEAK | BETTER | BETTER |

1. A food or beverage for a nonhuman animal, comprising: a sesamin-class compound(s) of 1 wt % or above, and an additive acceptable as a food or beverage for an animal.

2. A food or beverage for a nonhuman animal to treat a coat and/or a body odor of the nonhuman animal, the food or beverage comprising: an effective amount of a sesamin-class compound(s) needed to treat the coat and/or the body odor of the nonhuman animal, and an additive acceptable as a food or beverage for an animal.

3. The food or beverage of claim 1, wherein the sesamin-class compound(s) are sesamin and/or episesamin.

4. The food or beverage of claim 1 further comprising a fat or an oil.

5. The food or beverage of claim 1 each of which is in a form of a supplement.

6. A method for treating a coat and/or a body odor of a nonhuman animal, comprising administering to the nonhuman animal the food or beverage as defined in claim 1.

7. A method for treating a coat and/or a body odor of a nonhuman animal, comprising administering to the nonhuman animal an effective amount of a sesamin-class compound(s) needed to treat the coat and/or the body odor of the nonhuman animal.

8. The method of claim 7, wherein the effective amount is not less than 0.15 mg per 1 kg of body weight of the nonhuman animal per day.

9. The method of claim 6, wherein the nonhuman animal is a pet.
